(19) United States
(12) Patent Application Publication
(10) Pub. No.: US 2001/0005746 A1
(43) Pub. Date: Jun. 28, 2001

(54) Stomatin-like genes and their use in plants

(75) Inventors: William J. Gordon-Kamm, Urbandale, IA (US); Keith S. Lowe, Johnston, IA (US); Ramgopal Nadimpalli, Des Moines, IA (US); Carl R. Simmons, Des Moines, IA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Hi-Bred International, Inc., Des Moines, IA

(21) Appl. No.: 09/767,129

(22) Filed: Jan. 22, 2001

Related U.S. Application Data
(62) Division of application No. 09/395,397, filed on Sep. 14, 1999.
(60) Provisional application No. 60/100,748, filed on Sep. 17, 1998.

(51) Int. Cl. C07K 14/415; C07H 21/04

(52) U.S. Cl. 530/370; 536/23.6

(57) ABSTRACT

Compositions and methods for enhancing disease resistance in plants are provided. The method involves transforming a plant with an stomatin-like sequence. The stomatin-like sequence acts to induce the plant defense response in the plant cell. A pathogen inducible promoter or alternatively a constitutive, preferably a weak constitutive, promoter is used to control the desired level of disease control in the plant.

Transformed plants, plant cells, tissues, and seed are also provided having enhanced disease resistance.

### CROSS-REFERENCE TO RELATED APPLICATION

This application is a divisional application of U.S. patent application Ser. No. 09/395,397, filed Sep. 14, 1999, which claims the benefit of U.S. Provisional Application No. 60/100,748, filed on Sep. 17, 1998.

### FIELD OF THE INVENTION

The invention relates to the genetic manipulation of plants, particularly to transforming plants with genes that are involved in ion transport, cellular division, and disease resistance.

### BACKGROUND OF THE INVENTION

Stomatin is an integral membrane protein found in red blood cells. The protein migrates at 31 kDa on SDS gels. Hereditary stomatocytosis is a rare hemolytic anemia characterized by abnormal membrane permeability to univalent cations. In the most severe form of the disease, overhydrated hereditary stomatocytosis, stomatin is essentially absent. The disease is characterized by the leaking of univalent cations by the plasma membrane of red blood cells. The red blood cells show abnormal permeability to Na+ and K+. It is hypothesized that there is an ion leak in the erythrocytes, with an inadequate compensatory increase in transport by the Na+/K+ pump.

The cDNA for the mammalian protein has been cloned. The novel protein is not homologous to known membrane proteins and is believed to act as a trans-acting cation channel regulator. While the molecular mechanism for stomatin function is unknown, the concomitant leak and absence of protein suggest that the protein may act as a “plug” for ion channels in normal cells. The protein may support, activate, or regulate an as yet unidentified associated ion channel.

Disease in plants is caused by biotic and abiotic causes. Biotic causes include fungi, viruses, bacteria, and nematodes. Of these, fungi are the most frequent causative agent of disease on plants. Abiotic causes of disease in plants include extremes of temperature, water, oxygen, soil pH, plus nutrient-element deficiencies and imbalances, excess heavy metals, and air pollution.

A host of cellular processes enables plants to defend themselves from disease caused by pathogenic agents. These processes apparently form an integrated set of resistance mechanisms that is activated by initial infection and then limits farther spread of the invading pathogenic microorganism.

As noted, among the causative agents of infectious disease of crop plants, the phytopathogenic fungi play the dominant role. Phytopathogenic fungi cause devastating epidemics, as well as causing significant annual crop yield losses. All of the approximately 300,000 species of flowering plants are attacked by pathogenic fungi. However, a single plant species can be host to only a few fungal species, and similarly, most fungi usually have a limited host range.

Plant disease outbreaks have resulted in catastrophic crop failures that have triggered famines and caused major social change. Generally, the best strategy for plant disease control is to use resistant cultivars selected or developed by plant breeders for this purpose. However, the potential for serious crop disease epidemics persists today, as evidenced by outbreaks of the Victoria blight of oats and southern corn leaf blight. Accordingly, molecular methods are needed to supplement traditional breeding methods to protect plants from pathogen attack.

### SUMMARY OF THE INVENTION

A stomatin-like gene from plants is provided. The gene bears homology to hypersensitive response (HR) inducing genes. The sequences of the invention may thus find use in the activation of the plant pathogen defense system. Hence,the compositions and methods of the invention can be used for enhancing resistance to plant pests. The method involves stably transforming a plant with a nucleotide sequence capable of inducing the plant pathogen defense system operably linked with a promoter capable of driving expression of a gene in a plant cell.

The stomatin-like sequences additionally find use in manipulating ion transport in transformed plants and plant cells. Transformed plants and seeds, as well as methods for making such plants and seeds are additionally provided.

It is recognized that a variety of promoters will be useful in the invention, the choice of which will depend in part upon the desired level of expression of the disclosed genes. It is recognized that the levels of expression can be controlled to induce the disease resistance pathway resulting in levels of immunity in the plant which impart resistance in the plant to the pathogen or to induce cell death.

The methods of the invention find use in controlling plant pests, including fungal pathogens, viruses, nematodes, insects, and the like.

### BRIEF DESCRIPTION OF THE DRAWINGS

FIG. 1 schematically illustrates the plasmid construct comprising the ubiquitin promoter and a stomatin sequence.

FIG. 2 schematically illustrates the plasmid construct comprising the ubiquitin promoter and CRC fusion protein gene.

### DETAILED DESCRIPTION OF THE INVENTION

Plant genes having sequence homology to stomatins are provided. The “stomatin-like” sequences also show homology to hypersensitive response-inducing genes. Such genes may find use in altering ion transport and for inducing disease and pathogen resistance in a plant. Accordingly, the compositions of the invention find use in protecting plants against fungal pathogens, viruses, nematodes, insects and the like.

Compositions of the invention include a stomatin protein that may be involved in ion transport and plant disease resistance. In particular, the present invention provides for an isolated nucleic acid molecules comprising nucleotide sequences encoding the amino acid sequence shown in SEQ ID NO:2, or the nucleotide sequence encoding the DNA sequence deposited in a bacterial host as Patent Deposit No. 98871. Further provided is a polypeptide having an amino acid sequence encoded by a nucleic acid molecule described herein, for example that set forth in SEQ ID NO: 1, that deposited in a bacterial host as Patent Deposit No. 98871 and fragments and variants thereof.

A plasmid containing the nucleotide sequences of the invention were deposited with the Patent Depository of the American Type Culture Collection (ATCC), Manassas, Va., and assigned Patent Deposit No. 98871. This deposit will be maintained under the terms of the Budapest Treaty on the International Recognition of the Deposit of Microorganisms for the Purposes of Patent Procedure. This deposit was made merely as a convenience for those of skill in the art and are not an admission that a deposit is required under 35 U.S.C. §112.

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed nucleotide sequence and the protein encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence retain stomatin-like activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of a stomatin nucleotide sequence that encodes a biologically active portion of a stomatin protein of the invention will encode at least 15, 25, 30, 50, 100, 150, 200, 250, 300, 350 contiguous amino acids, or up to the total number of amino acids present in a full-length stomatin protein of the invention (for example, 394 amino acids for SEQ ID NO: 2. Fragments of a stomatin nucleotide sequence that are useful as hybridization probes for PCR primers generally need not encode a biologically active portion of a stomatin protein.

Thus, a fragment of a stomatin nucleotide sequence may encode a biologically active portion of a stomatin, or it may be a fragment that can be used as a hybridization probe or PCR primer using methods disclosed below. A biologically active portion of a stomatin can be prepared by isolating a portion of the stomatin nucleotide sequences of the invention, expressing the encoded portion of the stomatin protein (e.g., by recombinant expression in vitro), and assessing the activity of the encoded portion of the stomatin protein. Nucleic acid molecules that are fragments of a stomatin nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, or 1,400, 1,500, 1,600 nucleotides, or up to the number of nucleotides present in a full-length 1,662 nucleotide sequence disclosed herein (for example, 1662 nucleotides for SEQ ID NO: 1.

By “variants” is intended substantially similar sequences. For nucleotide sequences, conservative variants include those sequences that, because of the degeneracy of the genetic code, encode the amino acid sequence of the stomatin polypeptide of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode the stomatin protein of the invention. Generally, variants of a particular nucleotide sequence of the invention will have at least 40%, 50%, 60%, 70%, generally at least 75%, 80%, 85%, preferably about 90% to 95% or more, and more preferably about 98% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, stomatin-like activity as described herein. Such variants may result from, for example, genetic polymorphism or from human manipulation. Biologically active variants of a native stomatin protein of the invention will have at least 40%, 50%, 60%, 70%, generally at least 75%, 80%, 85%, preferably about 90% to 95% or more, and more preferably about 98% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the stomatin protein can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired stomatin-like activity. Obviously, the mutations that will be made in the DNA encoding the variant must not place the sequence out of reading frame and preferably will not create complementary regions that could produce secondary mRNA structure. See, EP Patent Application Publication No. 75,444.

The deletions, insertions, and substitutions of the protein sequence encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by the induction of the plant defense system. See, for example U.S. Pat. No. 5,614,395, herein incorporated by reference. Additionally, the activity can also be evaluated by a decrease in cell division rate. This decrease in division rate is reflected in larger cell size, less rapid incorporation of radiolabeled nucleotides and slower growth. See for example, Nuell, M. J. et al. (1991) Molecular and Cellular Biology 11:1372-1381, and Roskams, J. A. et al. (1993) Journal of Cellular Physiology 157:289-295.

Variant nucleotide sequences and proteins also encompass sequences and proteins derived from a mutagenic and recombinogenic procedure such as DNA shuffling. With such a procedure, one or more different stomatin coding sequences can be manipulated to create a new stomatin possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the stomatin gene of the invention and other known stomatin genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants, more particularly other monocots. In this manner, methods such as PCR, hybridization, and the like can be used to identify such sequences based on their sequence homology to the sequence set forth herein. Sequences isolated based on their sequence identity to the entire stomatin sequence set forth herein or to fragments thereof are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the stomatin sequence of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire stomatin sequence disclosed herein, or one or more portions thereof, may be used as a probe capable of specifically hybridizing to corresponding stomatin sequences and messenger RNAs. To achieve specific hybridization under a variety of conditions, such probes include sequences that are unique among stomatin sequences and are preferably at least about 10 nucleotides in length, and most preferably at least about 20 nucleotides in length. Such probes may be used to amplify corresponding stomatin sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× X to 2× SSC (20× SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1× SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, N.Y.). See Sambrook et al. (1989) Molecular Cloning: A Laboratory Manual (2d ed., Cold Spring Harbor Laboratory Press, Plainview, N.Y.).

Thus, isolated sequences that encode for a stomatin protein and which hybridize to the stomatin sequence disclosed herein, or to fragments thereof, are encompassed by the present invention. Such sequences will be at least 40% to 50% homologous, about 60% to 70% homologous, and even about 80%, 85%, 90%, 95% to 98% homologous or more with the disclosed sequence. That is, the sequence identity of sequences may range, sharing at least about 40% to 50%, about 60% to 70%, and even about 80%, 85%, 90%, 95% to 98% sequence identity.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman et al. (1970) J. Mol. Biol. 48:443; the search-for-similarity-method of Pearson et al. (1988) Proc. Natl. Acad. Sci. 85:2444; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman et al. (1970) J. Mol. Biol. 48:443. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

Stomatin is an integral membrane protein believed to function as a negative regulator of univalent cation permeability. The cytoplasmic portion of the protein has been suggested to act as a ball and chain tether that can directly plug ion channels. Thus, the sequences of the invention may find use in modulating ion transport processes in physiology, in signaling, and in the control of the volume of both intracellular and extracellular compartments.

While the invention is not bound by any mechanism, it is recognized that the stomatin-like protein may function by linking with another protein or proteins. Such additional proteins may be elucidated by methods known in the art. See, for example, Sambrook et al (1989) Molecular Cloning: A Laboratory Manual (2d. ed., Cold Spring Harbor Laboratory Press, Plainview, N.Y.); Innis et al. (1990) PCR Protocols: A Guide to Methods and Applications (Academic Press, New York); Plant Molecular Biology: A Laboratory Manual (1997) Melody S. Clark (Ed.), Springer, N.Y.; Current Protocols in Molecular Biology (1998) Ausubel et al. (Eds.), John Wiley & Sons, Inc.; Langenheim and Thimann, (1982) Botany: Plant Biology and Its Relation to Human Affairs (John Wiley); Vasil, ed. (1984) Cell Culture and Somatic Cell Genetics of Plants, Vol. 1; Stanier et al. (1986) The Microbial World (5th ed., Prentice-Hall); Dhringra and Sinclair (1985) Basic Plant Pathology Methods (CRC Press); Maniatis et al. (1982) Molecular Cloning: A Laboratory Manual (Cold Spring Harbor Laboratory Press, Cold Spring Harbor, N.Y.); Glover, ed. (1985) DNA Cloning, Vols. I and II; Gait, ed. (1984) Oligonucleotide Synthesis; Hames and Higgins, eds. (1984) Nucleic Acid Hybridization; the series Methods in Enzymology (Colowick and Kaplan, eds., Academic Press, Inc.); Davis et al., eds. (1980) Advanced Bacterial Genetics (Cold Spring Harbor Laboratory, Cold Spring Harbor, N.Y.); and the references cited therein, all of which are herein incorporated by reference.

The stomatin sequences of the invention form part of a structurally-related family of proteins. Prohibitins and HR-inducing genes are also members of this family. See U.S. patent application entitled “Maize Prohibitin Genes and Methods of Use” filed concurrently herewith and U.S. patent application Ser. No. 09/256,158 filed on Feb. 24, 1999, entitled “Genes for Activation of Plant Pathogen Defense Systems.” The stomatin sequences of the invention have “stomatin-like activity”. Examples of stomatin-like activity include the control of ionic states. In addition, the control of ionic states by stomatins may control defense signaling and/or cell cycle. Thus, the sequences of the invention may find use in controlling cell cycle, cell proliferation and in disease resistance.

By “disease resistance” is intended that the plants avoid the disease symptoms which are the outcome of plant-pathogen interactions. That is, pathogens are prevented from causing plant diseases and the associated disease symptoms, or alternatively, the disease symptoms caused by the pathogen is minimized or lessened. The methods of the invention can be utilized to protect plants from disease, particularly those diseases that are caused by plant pathogens.

The genes of the invention include those which are involved in activating plant systems for defense against pathogens. Because of the homology to pathogen resistance genes, it is believed that the gene products, probably proteins or polypeptides, function to activate the defense system. The activation of the defense system may involve inducing resistance of the plant to pathogen invasion or alternatively, the gene products may turn on the hypersensitive response in the plant. The hypersensitive response is a localized lesion of necrotic tissue that forms around the site of pathogen infection. This plant-induced necrosis thwarts progression of the disease by depriving the pathogen of plant material for its consumption.

Alternatively, activation of the plant defense system may involve the induced production of gene products, such as PR proteins and various secondary metabolites, many of which are antipathogenic. The induction may involve inducing the accumulation of cytotoxic phytoalexins, the deposition of callose and lignin in cell walls. Likewise, the induction may involve the activation of transcription factors, reactive oxygen species, ion fluxes, G proteins, salicylic acids and other HR and plant defense regulators. It is recognized that the present invention is not dependent upon a particular mechanism of defense. Rather, the genes and methods of the invention work to increase resistance of the plant to pathogens independent of how that resistance is brought.

As discussed, the expression of the stomatin-like molecules in the plant cell induces the disease resistance pathway or induces immunity, i.e., disease resistance, in the plant. That is, the expression of the genes can induce a defense response in the cell or can turn on the disease resistance pathway to obtain cell death. The end result can be controlled by the level of expression of the stomatin-like sequences in the plant. Where the expression is sufficient to cause cell death, such response is a receptor-mediated programmed response. See, for example, Ryerson and Heath (1996) Plant Cell 8:393-402 and Dangl et al. (1996) Plant Cell 8:1793-1807.

The methods of the invention can be used with other methods available in the art for enhancing disease resistance in plants. The stomatin-like molecules described herein may be used alone or in combination with other proteins or agents to protect against plant diseases and pathogens. Other plant defense proteins include those described in U.S. application Ser. No. 09/256,898 “Methods for Enhancing Disease Resistance in Plants” filed on Feb. 24, 1999.

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, maize dwarf mosaic virus, etc. Specific fungal and viral pathogens for the major crops include: Soybeans: Phytophthora megasperma f. sp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium roltsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, Amnerican Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Nematodes include parasitic nematodes such as root-knot, cyst, and lesion nematodes, including Heterodera and Globodera spp; particularly Globodera rostochiensis and globodera pailida (potato cyst nematodes); Heterodera glycines (soybean cyst nematode); Heterodera schachtii (beet cyst nematode); and Heterodera avenae (cereal cyst nematode).

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm ; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Phopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Franklinkiella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall army; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcom maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

In addition, the sequences of the invention may provide a means of modulating the cell cycle particularly by manipulation of ion transport. That is, the sequences may be used to block or reduce cell cycle arrest and thus increase transformation frequency. Besides increasing transformation efficiency in plants, the methods find use in transforming genotypes of plant species recalcitrant to transformation. Generally, all methods of plant transformation yield low transformation frequency. Thus, the methods of the invention can be used with any transformation method including Agrobacterium infection, electroporation, protoplast fusion, particle bombardment, and the like.

As stomatins may control cell cycle through ionic states, expression may lead to inhibition of the cell cycle and/or cell proliferation. Thus, to increase cell division, inhibition of stomatin may be desired. This increase in cell division may be brought about by expression of the stomatin sequence or interference with the stomatin gene product. Any method for suppression of expression can be used in the invention. Such methods are known in the art and include antisense constructions, cosuppression, protein and mRNA manipulation, use of nucleotide or ribonucleotide sequences, antibodies, proteins, peptides, and the like. Over-expression of the stomatin sequence may lead to co-suppression. Antisense sequences may be used to down regulate expression. Antibodies or proteins may be used to transiently disrupt the growth inhibitory properties of stomatin. Likewise, contransformation with ribo- or deoxyribo-oligonucleotides based on stomatin sequences may be used to down regulate expression.

The delivery of nucleotides, proteins and antibodies can be accomplished by indirect methods such as Agrobacterium or alternatively by direct methods such as electroporation, particle bombardment or sonication.

In methods to increase transformation efficiency, stomatin expression can be manipulated during and immediately following the introduction of the nucleotide sequences to be integrated into the plant genome. However, to avoid tumorous or uncontrolled growth, the alteration of cell division will need to be limited. To avoid problems associated with ectopic stable expression of the sequences of the invention, strategies for transient expression of the sequences may be needed. In this manner, as noted above, delivery of peptides, DNA, RNA, or antibodies could be used to enhance transgene integration by transient stimulation of cell division. For example, inactivation of Rb or stomatin protein with antibodies or antisense RNA would work to drive cells into S-phase.

By “increasing transformation efficiency” is intended that the number of transformed plants obtained from a single transformation event is increased at least 1, 2, 3 fold to 10 fold or more.

The sequences and methods of the invention may also find use in initiating tissue cultures in plant genotypes recalcitrant to culturing and/or transformation. Cell division rates in maize and other plants affect both the culture initiation frequencies and transformation frequencies of the plant. In some genotypes it is extremely difficult to stimulate cell proliferation in vitro. This lack of adequate culture response has a negative effect on transformation as cell culture is required for most transformation methods. Thus, methods of the invention can be used to stimulate cell division and initiate plant tissue cultures.

The methods find use in initiating cell cultures or increasing transformation in any plant of interest. Plants of interest include, but are not limited to corn (Zea mays), canola (Brassica napus, Brassica rap ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats, barley, vegetables, ornamentals, and conifers. Preferably plants include corn, soybean, sunflower, safflower, Brassica, wheat, barley, rye, alfalfa, and sorghum.

The present invention additionally provides a method of genotyping a plant comprising a polynucleotide of the present invention. Preferably, the plant is a monocot, such as maize or sorghum. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson 1996 (Chapter 2) in: Genome Mapping in Plants (ed. Andrew H. Paterson) by Academic Press/R. G. Landis Company, Austin, Tex., pp.7-21.

The particular method of genotyping in the present invention may employ any number of molecular marker analytic techniques such as, but not limited to, restriction fragment length polymorphisms (RFLPs). RFLPs are the product of allelic differences between DNA restriction fragments caused by nucleotide sequence variability. As is well known to those of skill in the art, RFLPs are typically detected by extraction of genomic DNA and digestion with a restriction enzyme. Generally, the resulting fragments are separated according to size and hybridized with a probe; single copy probes are preferred. Restriction fragments from homologous chromosomes are revealed. Differences in fragment size among alleles represent an RFLP. Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, preferably within 20 or 10 cM, more preferably within 5, 3, 2, or 1 cM of a gene of the invention.

In the present invention, the nucleic acid probes employed for molecular marker mapping of plant nuclear genomes selectively hybridize, under selective hybridization conditions, to a gene encoding a polynucleotide of the present invention. In preferred embodiments, the probes are selected from polynucleotides of the present invention. Typically, these probes are cDNA probes or Pst I genomic clones. The length of the probes is discussed in greater detail, supra, but are typically at least 15 bases in length, more preferably at least 20, 25, 30, 35, 40, or 50 bases in length. Generally, however, the probes are less than about 1 kilobase in length. Preferably, the probes are single copy probes that hybridize to a unique locus in a haploid chromosome complement. Some exemplary restriction enzymes employed in RFLP mapping are EcoRI, EcoRv, and SstI. As used herein the term “restriction enzyme” includes reference to a composition that recognizes and, alone or in conjunction with another composition, cleaves at a specific nucleotide sequence.

The method of detecting an RFLP comprises the steps of (a) digesting genomic DNA of a plant with a restriction enzyme; (b) hybridizing a nucleic acid probe, under selective hybridization conditions, to a sequence of a polynucleotide of the present of said genomic DNA; (c) detecting therefrom a RFLP. Other methods of differentiating polymorphic (allelic) variants of polynucleotides of the present invention can be had by utilizing molecular marker techniques well known to those of skill in the art including such techniques as: 1) single stranded conformation analysis (SSCP); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. Other approaches based on the detection of mismatches between the two complementary DNA strands include clamped denaturing gel electrophoresis (CDGE); heteroduplex analysis (HA); and chemical mismatch cleavage (CMC). Exemplary polymorphic variants are provided in Table I, supra. Thus, the present invention further provides a method of genotyping comprising the steps of contacting, under stringent hybridization conditions, a sample suspected of comprising a polynucleotide of the present invention with a nucleic acid probe. Generally, the sample is a plant sample; preferably, a sample suspected of comprising a maize polynucleotide of the present invention (e.g., gene, mRNA). The nucleic acid probe selectively hybridizes, under stringent conditions, to a subsequence of a polynucleotide of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In preferred embodiments, the nucleic acid probe comprises a polynucleotide of the present invention.

The stomatin-like genes of the invention can be introduced into any plant. The genes to be introduced can be conveniently used in expression cassettes for introduction and expression in any plant of interest.

Such expression cassettes will comprise a transcriptional initiation region linked to the stomatin-like sequence of interest. Such an expression cassette is provided with a plurality of restriction sites for insertion of the gene of interest to be under the transcriptional regulation of the regulatory regions. The expression cassette may additionally contain selectable marker genes.

The transcriptional initiation region, the promoter, may be native or analogous or foreign or heterologous to the plant host. Additionally, the promoter may be the natural sequence or alternatively a synthetic sequence. By foreign is intended that the transcriptional initiation region is not found in the native plant into which the transcriptional initiation region is introduced. As used herein a chimeric gene comprises a coding sequence operably linked to a transcription initiation region that is heterologous to the coding sequence.

While it may be preferable to express the sequences using heterologous promoters, the native promoter sequences may be used. Such constructs would change expression levels of stomatin in the plant or plant cell. Thus, the phenotype of the plant or plant cell is altered.

The expression cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source. Convenient termination regions are available from the Ti-plasmid of A. tumefaciens, such as the octopine synthase and nopaline synthase termination regions. See also, Guerineau et al., (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

The genes of the invention are provided in expression cassettes for expression in the plant of interest. The cassette will include 5′ and 3′ regulatory sequences operably linked to the gene of interest. The cassette may additionally contain at least one additional gene to be cotransformed into the organism. Alternatively, the additional gene(s) can be provided on another expression cassette. Where appropriate, the gene(s) may be optimized for increased expression in the transformed plant. That is, the genes can be synthesized using plant preferred codons for improved expression. See, for example, Campbell and Gowri (1990) Plant Physiol. 92:1-11 for a discussion of host-preferred codon usage. Methods are available in the art for synthesizing plant preferred genes. See, for example, U.S. Pat. Nos. 5,380,831, 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences which may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94; untranslated leader from the coat protein mRNA of alfalfa mosaic virus (AMV RNA 4), (Jobling et al. (1987) Nature 325:622-625; tobacco mosaic virus leader (TMV), (Gallie et al. (1989) Mole. Biol. of RNA, pages 237-256; and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Towards this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions, may be involved.

A number of promoters can be used in the practice of the invention. The promoters can be selected based on the desired outcome. When the genes are expressed at levels to causes cell death, an inducible promoter can be used to drive the expression of the genes of the invention. The inducible promoter must be tightly regulated to prevent unnecessary cell death yet be expressed in the presence of a pathogen to prevent infection and disease symptoms. Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) The Plant Cell 4:645-656; and Van Loon (1985) Plant Mol. Virol. 4:111-116. See, also U.S. patent application Ser. No. 09/257,583 entitled “Constitutive Maize Promoters” filed on Feb. 25, 1999, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Molecular and General Genetics 2:93-98; and Yang, Y (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; and the references cited therein. Of particular interest is the inducible promoter for the maize PRms gene, whose expression is induced by the pathogen Fusarium moniliforme (see, for example, Cordero et al. (1992) Physiological and Molecular Plant Pathology 41:189-200).

Additionally, as pathogens find entry into plants through wounds or insect damage, a wound inducible promoter may be used in the constructions of the invention. Such wound inducible promoters include potato proteinase inhibitor (pin II) gene (Ryan, C., Annu. Rev. Phytopath 28:425-449; Duan et al. Nature Biotechnology 14:494-498); wun1 and wun2, U.S. Pat. No. 5,428,148; win1 and win2 (Stanford et al Mol Gen Genet 215:200-208); systemin (McGurl et al. Science 225:1570-1573); WIP1(Rohmeier et al. Plant Mol. Biol. 22:783-792; Eckelkamp et al. FEBS Letters 323:73-76); MPI gene (Corderok et al. The Plant Journal 6(2):141-150); and the like, herein incorporated by reference.

Where low level expression is desired, weak promoters will be used. It is recognized that weak inducible promoters may be used. Additionally, either a weak constitutive or a weak tissue-preferred promoter may be used. Such weak promoters cause activation of the plant defense system short of hypersensitive cell death. Thus, there is an activation of the plant defense system at levels sufficient to protect from pathogen invasion. In this state, there is at least a partial activation of the plant defense system wherein the plant produces increased levels of antipathogenic factors such as PR proteins, i.e., PRI, chitinases, β-glucanases, etc.; secondary metabolites; phytoalexins; reactive oxygen species; and the like.

Generally, by “weak promoter” is intended either a promoter that drives expression of a coding sequence at a low level. By low level is intended at levels of about 1/1000 transcripts per cell to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels.

Such weak constitutive promoters include, for example, the core promoter of the Rsyn7 (PCT Application Ser. No. US99/03863), the core 35S promoter, and the like. Other constitutive promoters include, for example, U.S. Pat. Nos. 5,608,149; 5,608,144; 5,604,121; 5,569,597; 5,466,785; 5,399,680; 5,268,463; 5,608,142. See also, copending application entitled “Constituative Maize Promoters” Ser. No. 60/076,075, filed Feb. 26, 1998 and herein incorporated by reference.

Tissue-preferred promoters include Yamamoto et al. (1997) Plant J. 12(2):255-265; Kawamata et al. (1997) Plant Cell Physiol. 38(7):792-803; Hansen et al. (1997) Mol Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc. Natl. Acad. Sci. U.S.A. 90(20):9586-9590; and Guevara-Garcia et al. (1993) Plant J. 4(3):495-505. Such promoters can be modified, if necessary, for weak expression.

As used herein, “antisense orientation” includes reference to a duplex polynucleotide sequence which is operably linked to a promoter in an orientation where the antisense strand is transcribed. The antisense strand is sufficiently complementary to an endogenous transcription product such that translation of the endogenous transcription product is often inhibited.

A polynucleotide of the present invention can be expressed in either sense or anti-sense orientation as desired. It will be appreciated that control of gene expression in either sense or anti-sense orientation can have a direct impact on the observable plant characteristics. Antisense technology can be conveniently used to control gene expression in plants. To accomplish this, a nucleic acid segment from the desired gene is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy et al., Proc. Nat'l. Acad. Sci. (USA) 85:8805-8809 (1988); and Hiatt et al., U.S. Pat. No. 4,801,340.

Antisense nucleotides of the stomatin sequences are complementary to at least a portion of the stomatin messenger RNA (mRNA). The expressed nucleotide sequences hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence similarity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988).

While novel nucleotide sequences are disclosed, it is recognized that sequences from other sources, including mammalian sources, may be used in the practice of the invention. Such nonplant sequences can be constructed using plant preferred codons if necessary for expression in plants. Additionally, promoters capable of driving expression of the sequences in plants can be used with such sequences.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055), direct gene transfer (Paszkowski et al. (1984) EMBO J 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The present invention may be used for transformation of any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

The cells that have been transformed may be grown into plants in accordance with conventional ways. See, for example, McCormick et al. (1986) Plant Cell Reports, 5:81-84. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

Generally, methods for antibody production are known in the art. The antibodies of the invention selectively bind to the stomatin protein and its variants and fragments. An antibody is considered to selectively bind, even if it also binds to other proteins that are not substantially homologous with the stomatin protein. These other proteins share homology with a fragment or domain of the stomatin protein. This conservation in specific regions gives rise to antibodies that bind to both proteins by virtue of the homologous sequence. In this case, it would be understood that antibody binding to the stomatin protein is still selective. Antibodies can be polyclonal, or more preferably, monoclonal. An intact antibody, or a fragment thereof (e.g. Fab or F(ab′)2) can be used.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵U.S. or ³H.

To generate antibodies, an isolated receptor polypeptide is used as an inumunogen to generate antibodies using standard techniques for polyclonal and monoclonal antibody preparation. Either the full-length protein or antigenic peptide fragment can be used. Preferably, the antigenic peptide comprises at least 10 amino acid residues, more preferably at least 15 amino acid residues, even more preferably at least 20 amino acid residues, and most preferably at least 30 amino acid residues. An appropriate immunogenic preparation can be derived from native, recombinantly expressed, protein or chemically synthesized peptides.

The following examples are offered by way of illustration and not by way of limitation.

### EXPERIMENTAL

### EXAMPLE 1

### Transient Gene Expression Assay Using Biolistics Particle Bombardment

A transient gene expression assay, as modified from Nelson and Bushnell (Transgenic Res. (1997) 6:233-244), is used to evaluate the ability of a polypeptide encoded by a stomatin-like gene, to induce the pathogen defense system in a host plant cell and confer a hypersensitive response within the host cell. In the method, a particle bombardment system is used to simultaneously introduce a construct comprising a reporter gene driven by a constitutive promoter and a construct comprising an stomatin-like gene operably linked to a promoter, into maize cells for the purposes of studying physiological processes, foremost amongst them the plant defense response.

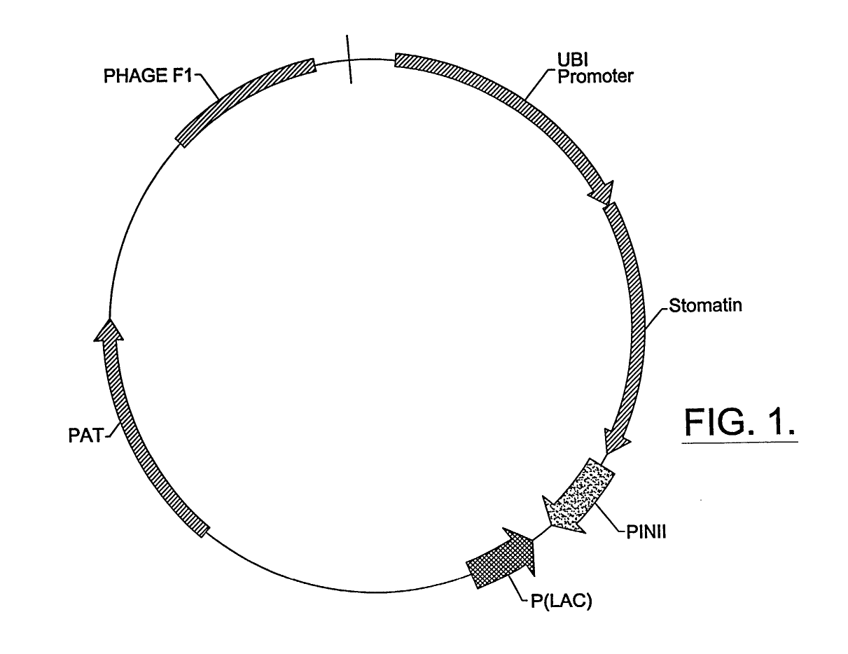

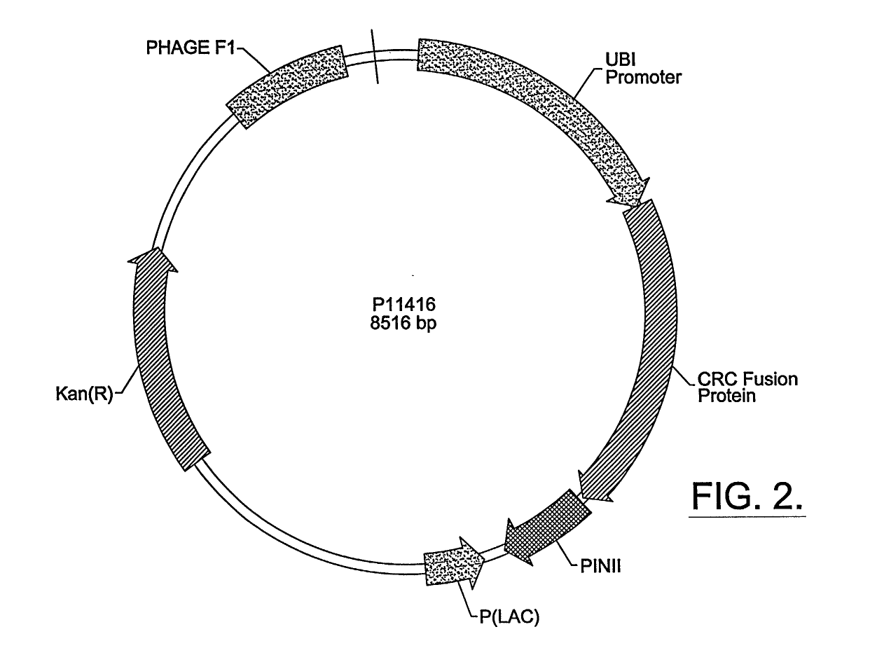

Immature maize embryos from greenhouse donor plants are bombarded with two plasmids. The first plasmid contains the stomatin nucleotide sequence operably linked to a ubiquitin promoter (FIG. 1). This plasmid also contains the selectable marker gene PAT (Wohlleben et al. (1988) Gene 70:25-37) that confers resistance to the herbicide Bialaphos. The second plasmid comprises a ubiquitin promoter operably linked to a nucleotide sequence encoding a reporter CRC fusion protein (FIG. 2). Expression of CRC causes the cells to turn red due to anthocyanin production. Other reporter genes, such as GUS, luciferase, or green fluorescent protein, can be used in this assay.

Transformation is performed as follows. All media recipes are in the Appendix.

Preparation of Target Tissue

The ears are surface sterilized in 30% Chlorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

The DNA constructs of interest used in this example include: a plasmid (FIG. 2), comprising the ubiquitin promoter (ubi) and the CRC fusion protein gene (ubi::CRC fusion), the expression of which yields the anthocyanin-producing, or red cell, phenotype; and a plasmid (FIG. 1), comprising the ubiquitin promoter and an stomatin-like sequence of the invention (ubi::stomatin-like sequence), the expression of which yields the stomatin-like product. Plasmid p7770 (not shown), comprising an empty ubiquitin promoter construct (ubi::pinII terminator), is used as a control to balance promoter site molarity; and plasmid p7731 (not shown), an inert DNA filler, is used to balance the amount of DNA shot with each bombardment episode.

The ubi::CRC fusion vector alone (Treatment A) is precipitated or the ubi::CRC vector and the ubi::stomatin-like vector are coprecipitated (Treatment B) onto 1.1 μm (average diameter) tungsten pellets using a CaCl₂ precipitation procedure as follows:

100 μl prepared tungsten particles in water

10 μl (1 μg) DNA in TrisEDTA buffer (1 μg total)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Expression of the CRC fusion gene is quantified by visual means 16 to 48 hours, more usually 36 hours, following bombardment. Cells expressing the CRC fusion protein gene are red in color. Assays are performed to determine if the expression of the stomatin nucleotide sequence in a cell causes a hypersensitive-type disease response involving cell death or at the very least radically redirects gene expression.

The activation of the defense system by the expression of the stomatin sequences is determined. Cell death disrupts the expression of the reporter gene, such that the occurrence of visible, anthocyanin-containing phenotypes is suppressed. Alternatively, the maize PR1 protein is used as a marker. An increased level of the maize PR-1 class of pathogenesis-related proteins is verified by Western blot analysis. Forty-eight hours after bombardment, 18 embryos from each treatment are pooled and their protein extracted and run on SDS-PAGE, electroblotted onto 0.2 micron PVDV membrane, and probed with antibodies raised against tobacco PR-1 protein.

### EXAMPLE 2

### Transient Stomatin-Antisense Expression Stimulates Cell Division and Enhances Transgene Integration

Regardless of the method of DNA delivery, cells competent for the integration of foreign DNA must be actively dividing. There is a growing body of evidence suggesting that integration of foreign DNA occurs in dividing cells (this includes both Agrobacterium and direct DNA delivery methods). It has long been observed that dividing transformed cells represent only a fraction of cells that transiently express a transgene. It is well known (in non-plant systems) that the delivery of damaged DNA, (similar to what we introduce by particle gun delivery methods) induces checkpoint controls and inhibits cell cycle progression. Cell cycle blockage may be regulated by proteins such as stomatin. This inhibition can be obviated by transient down-regulation of negative regulators such as stomatin. Regardless of the mechanism of arrest; i.e., presence of damaged DNA or delivery into a non-cycling differentiated cell, stimulation of the cell cycle will increase integration frequencies. To demonstrate this, a stomatin-antisense sequence is cloned into a cassette with a constitutive promoter (i.e., either a strong maize promoter such as the ubiquitin promoter including the first ubiquitin intron, or a weak constitutive promoter such as nos). Delivery of the stomatin-antisense DNA in an appropriate plant expression cassette (for example, in a UBI::Stomatin-antisense::pinII-containing plasmid) along with UBI::bar::pinII can be accomplished through numerous well-established methods for plant cells, including for example particle bombardment, sonication, PEG treatment or electroporation of protoplasts, electroporation of intact tissue, silica-fiber methods, microinjection or Agrobacterium-mediated transformation. Using one of the above methods, DNA is introduced into maize cells capable of growth on suitable maize culture medium. Such competent cells can be from maize suspension culture, callus culture on solid medium, freshly isolated immature embryos or meristem cells. Immature embryos of the Hi-II genotype are used as the target for co-delivery of these two plasmids. Transient expression of the stomatin-antisense down-regulates stomatin which in turn releases the cells to progress through the cell cycle and divide. This effectively overcomes the G1/S checkpoint controls, and increases the proportion of recipient-cells (i.e., into which DNA was introduced) that enter S-phase. This stimulation through the G1/S transition in cells harboring transgenic plasmid DNA provides an optimal cellular environment for integration of the introduced genes. Cytological methods can be used to verify increased frequencies of progression through S-phase and mitosis (i.e., for cells in which a visual marker such as GFP was transformed alongside stomatin the green fluorescent cells will exhibit a higher mitotic index). Cells in S-phase (undergoing DNA replication) can be monitored by detecting nucleotide analog incorporation. For example, following incubation of cells with bromodeoxyuridine (BrdU) incorporation of this thymidine analog can be detected by methods such as antiBrdU immunocytochemistry or through enhancement of Topro3 fluorescence following BrdU labeling. Stomatin expression will increase the proportion of cells incorporating BrdU (i.e., a higher percentage of transformed cells will incorporate BrdU relative to untransformed cells). Increased DNA synthesis can also be monitored using such methods as fluorescence activated cell sorting (FACS) of protoplasts (or nuclei), in conjunction with appropriate BrdU-insensitive fluorescent DNA labels such as propidium iodide and DAPI or BrdU-detecting methods described above. For example, tissue is homogenized to release nuclei that are analyzed using the FACS for both green fluorescence (from our accompanying GFP marker) and DNA content. Such FACS analysis demonstrates that expression of a co-transformed GFP reporter correlates with stomatin-induced changes in the ratios of cells in G1, S and G2. Similar experiments can be run using the fluorescently labeled anti-BrdU antisera to demonstrate that stomatin expression increased the percentage of cells in S-phase. Cell cycle stage-specific probes can also be used to monitor cell cycle progression. For example, numerous spindle-associated proteins are expressed during a fairly narrow window during mitosis, and antibodies or nucleic acid probes to cyclins, histones, or DNA synthesis enzymes can be used as positive markers for the G1/S transition. For cells that have received the stomatin-antisense gene cassette, stimulation of the cell cycle is manifested in an increased mitotic index, detected by staining for mitotic figures using a DNA dye such as DAPI or Hoechst 33258. FACS analysis of stomatin-antisense-expressing cells shows that a high percentage of cells have progressed into or through S-phase. Progression through S-phase will be manifested by fewer cells in G1 and more rapid cycling times (i.e., shorter G1 and G2 stages). A higher percentage of cells are labeled when cell cycle stage-specific probes are used, as mentioned above.

To assess the effect on transgene integration, growth of bialaphos-resistant colonies on selective medium is a reliable assay. Within 1-7 days after DNA introduction, the embryos are moved onto culture medium containing 3 mg/l of the selective agent bialaphos. Embryos, and later callus, are transferred to fresh selection plates every 2 weeks. After 6-8 weeks, transformed calli are recovered. Transgenic callus containing the introduced genes can be verified using PCR and Southern analysis. Northern analysis can also be used to verify which calli are expressing the bar gene, and/or the stomatin-antisense construct. In immature embryos that had transient, elevated stomatin-antisense expression, higher numbers of stable transformants are recovered (likely a direct result of increased integration frequencies). Increased transgene integration frequency can also be assessed using such well-established labeling methods such as in situ hybridization.

For this specific application (using transient stomatin-antisense-mediated cell cycle stimulation to increase transient integration frequencies), it may be desirable to reduce the likelihood of ectopic stable expression of stomatin-antisense. Strategies for transient-only expression can be used. This includes delivery of RNA (transcribed from the stomatin-antisense construct) along with the transgene cassettes to be integrated to enhance transgene integration by transient stimulation of cell division. Using well-established methods to produce stomatin-antisense-RNA, this can then be purified and introduced into maize cells using physical methods such as microinjection, bombardment, electroporation or silica fiber methods.

### EXAMPLE 3

### Use of Antisense Oligonucleotides Against Stomatin to Transiently Stimulate Cell Division and Enhances Transgene Integration

An alternative to conventional antisense strategies is the use of antisense oligonucleotides (often with chemically-modified nucleotides). Such an antisense oligonucleotide, typically a 15-18mer (but this size can vary either more or less), is designed to bind around accessible regions such as the ribosomal binding site around the “Start” codon. Introduction of the antisense oligonucleotide into a cell will transiently stop expression of the targeted gene. For example, an antisense oligonucleotide of between 15 to 18 nucleotides in length, that is complementary (in reverse orientation) to the sequence surrounding the Start codon of the stomatin structural gene, is introduced into maize cells. These methods of introduction for the oligonucleotide are similar to those previously described above for introduction of plasmids. In cells that receive such an antisense oligonucleotide targeted to stomatin, the antisense oligonucleotide transiently disrupts stomatin expression and stimulates entry into S-phase (as observed in mammalian cells—see Nuell et al., (1991) Mol. Cell. Biology 11(3):1372-1381).

### EXAMPLE 4

### Use of Antibodies Raised Against Stomatin to Transiently Stimulate Cell Division and Enhances Transgene Integration

Antibodies directed against stomatin can also be used to mitigate stomatin's tumor suppressor activity, thus stimulating the cell cycle and transgene integration. Genes encoding single chain antibodies, expressed behind a suitable promoter, for example the ubiquitin promoter, could be used in such a fashion. Transient expression of an anti-stomatin antibody could temporarily disrupt normal stomatin tumor suppressor function and thus stimulate the cell cycle. Alternatively, antibodies raised against stomatin could be purified and used for direct introduction into maize cells. The antibody is introduced into maize cells using physical methods such as microinjection, bombardment, electroporation or silica fiber methods. Alternatively, single chain anti-stomatin is delivered from Agrobacterium tumefaciens into plant cells in the form of fusions to Agrobacterium virulence proteins. Fusions are constructed between the anti-stomatin single chain antibody and bacterial virulence proteins such as VirE2, VirD2, or VirF which are known to be delivered directly into plant cells. Fusions are constructed to retain both those properties of bacterial virulence proteins required to mediate delivery into plant cells and the anti-stomatin activity required for stimulating cell division and enhancing transgene integration. This method ensures a high frequency of simultaneous co-delivery of T-DNA and functional anti-stomatin protein into the same host cell.

The methods above represent various means of using the stomatin-antisense or anti-stomatin antibodies, or antisense oligonucleotides to transiently stimulate DNA replication and cell division, which in turn enhances transgene integration by providing an improved cellular/molecular environment for this event to occur.

### EXAMPLE 5

### Altering Stomatin Expression Stimulates the Cell Cycle and Growth

Based on results in other eukaryotes, expression of the stomatin gene should block the G1/S transition and prevent cell division. This decrease in division rate is assessed in a number of different manners, being reflected in larger cell size, less rapid incorporation of radiolabeled nucleotides, and slower growth (i.e., less biomass accumulation). Conversely, expression of stomatin antisense (or an appropriate antisense oligonucleotide, or anti-stomatin antibody) will result in smaller cells, more rapid incorporation of radiolabeled nucleotides, and faster growth. Delivery of the stomatin-antisense in an appropriate plant expression cassette is accomplished through numerous well-established methods for plant cells, including for example particle bombardment, sonication, PEG treatment or electroporation of protoplasts, electroporation of intact tissue, silica-fiber methods, microinjection or Agrobacterium-mediated transformation. As an alternative to conventional deliver of bacterial plasmids, introduction of a viral plasmid from which a stomatin-antisense sequence is expressed could also be employed. The result of stomatin-antisense expression will be to stimulate the G1/S transition and hence cell division, providing the optimal cellular environment for integration of introduced genes. This will trigger a tissue culture response (cell divisions) in genotypes that typically do not respond to conventional culture techniques, or stimulate growth of transgenic tissue beyond the normal rates observed in wild-type (non-transgenic) tissues. To demonstrate this, the stomatin-antisense gene is cloned into a cassette with a constitutive promoter (i.e., either a strong maize promoter such as the ubiquitin promoter including the first ubiquitin intron, or a weak constitutive promoter such as nos). Either particle-mediated DNA delivery or Agrobacterium-mediated delivery are used to introduce the UBI::Stomatin::pinII-containing plasmid along with a UBI::bar::pinII-containing plasmid into maize cells capable of growth on suitable maize culture medium. Such competent cells can be from maize suspension culture, callus culture on solid medium, freshly isolated immature embryos or meristem cells. Immature embryos of the Hi-II genotype are used as the target for co-delivery of these two plasmids, and within 1-7 days the embryos are moved onto culture medium containing 3 mg/l of the selective agent bialaphos. Embryos, and later callus, are transferred to fresh selection plates every 2 weeks. After 6-8 weeks, transformed calli are recovered. In treatments where both the bar gene and stomatin-antisense gene have been transformed into immature embryos, a higher number of growing calli are recovered on the selective medium and callus growth is stimulated (relative to treatments with the bar gene alone). When the stomatin-antisense gene is introduced without any additional selective marker, transgenic calli can be identified by their ability to grow more rapidly than surrounding wild-type (non-transformed) tissues. Transgenic callus can be verified using PCR and Southern analysis. Northern analysis can also be used to verify which calli are expressing the bar gene, and which are expressing the maize stomatin gene at levels above normal wild-type cells (based on hybridization of probes to freshly isolated mRNA population from the cells).

Inducible Expression

The stomatin-antisense gene can also be cloned into a cassette with an inducible promoter such as the benzenesulfonamide-inducible promoter. The expression vector is co-introduced into plant cells and after selection on bialaphos, the transformed cells are exposed to the safener (inducer). This chemical induction of stomatin-antisense expression results in stimulated G1/S transition and more rapid cell division. The cells are screened for the presence of stomatin-antisense RNA by northern, or RT-PCR (using transgene specific probes/oligo pairs). Increased DNA replication is detected using BrdU labeling followed by antibody detection of cells that incorporated this thymidine analogue. Likewise, other cell cycle division assays could be employed, as described above.

### EXAMPLE 6

### Control of Stomatin-Antisense Expression Using Tissue-preferred or Cell-Specific Promoters Provides a Differential Growth Advantage

Stomatin-antisense expression using tissue-preferred or cell-specific promoters stimulates cell cycle progression in the expressing tissues or cells. For example, using a seed-specific promoter will stimulate cell division rate and result in increased seed biomass. Alternatively, driving stomatin-antisense expression with a strongly-expressed, early, tassel-specific promoter will enhance development of this entire reproductive structure. Expression of stomatin antisense in other cell types and/or at different stages of development will similarly stimulate cell division rates.

### EXAMPLE 7

### Meristem Transformation

Meristem transformation protocols rely on the transformation of apical initials or cells that can become apical initials following reorganization due to injury or selective pressure. The progenitors of these apical initials differentiate to form the tissues and organs of the mature plant (i.e., leaves, stems, ears, tassels, etc.). The meristems of most angiosperms are layered with each layer having its own set of initials. Normally in the shoot apex these layers rarely mix. In maize the outer layer of the apical meristem, the L1, differentiates to form the epidermis while descendents of cells in the inner layer, the L2, give rise to internal plant parts including the gametes. The initials in each of these layers are defined solely by position and can be replaced by adjacent cells if they are killed or compromised. Meristem transformation frequently targets a subset of the population of apical initials and the resulting plants are chimeric. If for example, 1 of 4 initials in the L1 layer of the meristem are transformed only ¼ of epidermis would be transformed. Selective pressure can be used to enlarge sectors but this selection must be non-lethal since large groups of cells are required for meristem function and survival. Transformation of an apical initial with a stomatin-antisense sequence under the expression of a promoter active in the apical meristem (either meristem-specific or constitutive) would allow the transformed cells to grow faster and displace wild-type initials driving the meristem towards homogeneity and minimizing the chimeric nature of the plant body. To demonstrate this, the stomatin-antisense sequence is cloned into a cassette with a promoter that is active within the meristem (i.e., either a strong constitutive maize promoter such as the ubiquitin promoter including the first ubiquitin intron, or a promoter active in meristematic cells such as the maize histone, cdc2 or actin promoter). Coleoptilar stage embryos are isolated and plated meristem up on a high sucrose maturation medium (see Lowe et al., 1997, In Genetic Biotechnology and Breeding of Maize and Sorghum, AS Tsaftaris, ed., Royal Society of chemistry, Cambridge, UK, pp94-97). The stomatin-antisense expression cassette along with a reporter construct such as Ubi:GUS:pinII can then be co-delivered (preferably 24 hours after isolation) into the exposed apical dome using conventional particle gun transformation protocols. As a control the stomatin-antisense construct can be replaced with an equivalent amount of pUC plasmid DNA. After a week to 10 days of culture on maturation medium the embryos can be transferred to a low sucrose hormone-free germination medium. Leaves from developing plants can be sacrificed for GUS staining. Transient expression of the stomatin-antisense sequence in meristem cells, through stimulation of the G1_S transition, will result in greater integration frequencies and hence more numerous transgenic sectors. Integration and expression of the stomatin-antisense sequence will impart a competitive advantage to expressing cells resulting in a progressive enlargement of the transgenic sector. Due to the enhanced growth rate in stomatin-antisense-expressing meristem cells, they will supplant wild-type meristem cells as the plant continues to grow. The result will be both enlargement of transgenic sectors within a given cell layer (i.e., periclinal expansion) and into adjacent cell layers (i.e., anticlinal invasions). As cells expressing the stomatin-antisense occupy an increasingly large proportion of the meristem, the frequency of transgene germline inheritance goes up accordingly.

### EXAMPLE 8

### Use of Flp/Frt System to Excise the Stomatin-Antisense Cassette

In cases where the stomatin-antisense has been integrated and stomatin-antisense expression is useful in the recovery of maize transgenics, but is ultimately not desired in the final product, the stomatin-antisense expression cassette (or any portion thereof that is flanked by appropriate FRT recombination sequences) can be excised using FLP-mediated recombination.

### EXAMPLE 9

Expressing the stomatin gene under the control of a cell-specific or tissue-preferred or a developmentally-regulated promoter will result in the cessation of growth in these cells or tissues. For example, using a tapetum-specific promoter or a microspore-specific promoter, expression of stomatin will result in aborted pollen development and male sterility. For certain uses such as hybrid production it may also be desirable to completely eliminate the male or female inflorescence. Expressing stomatin at early stages of ear or tassel development will result in failure of these organs to develop.

### EXAMPLE 10

### Identification of a Novel Protein Superfamily that Controls Cell Proliferation, Ion Channel Regulation, and Cell Death

A novel protein superfamily named PID, for Proliferation, Ion and Death, was identified. The new superfamily is comprised of three protein families, each respectively containing members involved in cell proliferation, ion conductance, and cell death. The members of the superfamily include many animal, bacteria, plant and fungi sequences representing prohibitins, NG1-like proteins (referred to as HIR proteins for Hypersensitive Induced Reaction), stomatins, and other membrane proteins. The structures of the superfamily members were analyzed with the goal of understanding their functional roles and molecular mechanisms that may explain and reconcile their involvement in apparently diverse physiological processes.

Methods

The HIR, prohibitin and stomatin homologues were identified with the aid of the IRIS software package from HGS, which includes the Blast algorithm. The alignment program indicated homology to tobacco NG1 (Genbank accession U66271), and to prohibitins from various species, and to human stomatin (Genbank accession U33925). Full-insert sequences were produced at Pioneer by forward and reverse sequencing and primer walking using an A.B.I. 377 sequencing machine. Sequences were assembled using Sequencher™ version 3.0 (Gene Codes Corporation, Ann Arbor, Mich.) and/or AssemblyLIGN™ (Eastman Kodak Company, New Haven, Conn.) software.

Initial database searches were carried out by BLASTP program (Altschul et al. (1990) J. Mol. Biol. 215:403-410) with chickpea HIR-like gene as a probe followed by PSI-BLAST (Altschul et al., (1997) Nucleic Acids Res. 25:3389-3402) with default parameters (Blosum 62, Gap existence cost 11, Per residue gap cost 1, Lambda ratio 0.85, Expect Threshold 10). About 32 sequences that appeared as significant hits, both in terms of statistical threshold and the type, were multiply aligned by ClustalW program with default parameters (Thompson et al., (1994) Nucleic Acids Res. 22:4673-4680).

Highly conserved residues were identified to generate a profile to perform further database searches by PHI-BLAST program (Zhang et al., (1998) Nucleic Acids Res. 26:3086-3990). Phylogenetic analysis was carried out by using the option within CLUSTALW (Higgins et al., (1996) Methods Enzymol 266:383-402) to generate multiple alignments followed by distance calculations and tree constructions with the PROTDIST and Neighbor-joining program of the PHYLIP package main.html—Felsenstein (Felsenstein (1993) PHYLIP (Phylogeny Inference Package) version 3.5c. Department of Genetics, University of Washington, Seattle). Pair-wise alignments were performed by GAP program within the GCG package (Felsenstein (1994) PHYLIP (Phylogeny Inference Package) version 8. Department of Genetics, University of Washington, Seattle).

After sufficient grounds were established for a possible evolutionary relationship among the sequences, further structural analyses were carried out notably by hydropathy profiles using Kyte-Doolittle method with a 19 residue sliding window (http://bioinformatics.weizmann.ac.il/hydroph/cmp_hydph.html). Secondary structure predictions were carried out by DSC algorithm (King et al. (1997) CABIOS 13:473-474). In order to look for conserved motifs in the 32 members included in the multiple alignment, we applied MEME algorithm which resulted in the detection of four highly conserved motifs (Grundy et al. (1997) Biochem. Biophys. Res. Commun. 231:760-766.

Results

Three distinct full-length maize cDNAs named Zm-HIR1, Zm-HIR2, and Zm-HIR3, for Zea mays Hypersensitive Induced Reaction genes one, two and three, respectively have been identified (U.S. patent application No. 09/256,158 filed on Feb. 24, 1999 entitled “Genes for Activation of Plant Defense Systems”) and share high homology to the tobacco NG1 peptide (Genbank accession U66271). Initial searches of maize HIR genes against the public database using the BLAST program (Altschul et al. (1990) J. Mol. Biol. 215:403-410) indicated some similarity to prohibitins and stomatins. For maize prohibitin sequences see U.S. Provisional Application entitled “Plant Prohibitin Genes and Their Use” filed concurrently herewith.

The coding region lengths for the HIR proteins (242-286 aa) are comparable to those of prohibitins (272-289 aa), and of stomatins and other membrane-associated proteins (249-481 aa). Pair-wise amino acid similarities of plant HIR and HIR-like genes with maize prohibitins were between 28-36% similar, and with maize stomatin between 34-37% similar. This suggested that the maize HIR genes were somewhat closer in amino acid sequence to stomatins than to prohibitins.

The non-redundant protein database at NCBI was searched using the PSI-BLAST program (Altschul et al. (1997) Nucleic Acids Res. 25:3389-3402) with a hypothetical protein from Chickpea (accession gi|3928150) as a probe, which has over 90% amino acid similarity with the maize HIR proteins. This search identified many genes, including stomatins and integral membrane proteins (E=<10⁻⁶) and prohibitins (E=<10⁻⁸), and HFLK/HFLC proteins (E=<10⁻⁶). These sequences were used to generate an unrooted dendogram which revealed a large superfamily with at least four constituent families. The stomatins and integral membrane proteins formed a large family that may possibly have some ion channel regulating activity in different organisms. A second family was composed of HIR and HIR-like sequences. The stomatin/integral membrane family was most closely related to this HIR family. The third family was composed of prohibitins and related sequences. The bacterial membrane proteins HFLK/HFLC formed a small fourth family more closely related to the prohibitin-containing family.

A comparison of hydropathy plots of maize HIR sequence with prohibitins from maize and Trypanosoma brucei revealed similar structural profiles. A similar comparison of a stomatin-like gene from Synchocystis sp. with Trypanosoma prohibitin also indicated structural similarity between several regions of these genes. The shared hydropathy plots indicate there are conserved structural features between these diverse proteins from widely diverged phyla, and thus suggests that the members at some level may possess a conserved function.

Amino acid sequences for 32 members of this superfamily were aligned to reveal shared and diverged features. Several regions are highly conserved and aligned well with fewer gaps. Two residues, an Aspartate and an Alanine, are completely conserved among all the proteins, and are located in the conserved secondary structural elements suggesting a critical role for these residues in the biological function of these proteins. The conserved Aspartate and Alanine residues in the stomatin amino acid sequence of SEQ ID NO:2 are located at amino acid positions 115 and 216, respectively.

A systematic search for conserved motifs in the 32 members of the superfamily was performed using the MEME algorithm. The MEME motifs have been indicated as reliable indicators of family membership (Grundy et al. (1997) Biochem. Biophys. Res. Commun. 231:760-766). The search resulted in the identification of four conserved motifs with a very high E-values. All four motifs are present in all members of HIR, somatin and prohibition families. Additionally, the localization of these four motifs in all these genes appears to be spatially well conserved, indicating the possibility for a similar structural orientation in three-dimensional space. We are unaware of any reports that members of this superfamily have been structurally resolved.

The HFLK/HFLC proteins contain only Motif 3. Hydropathy analysis also indicated that these genes have fewer structural similarities in common with other members in this superfamily. The HFLK/HFLC are bacterial membrane proteins with protease activity, and are involved in lysogenization. They appear to be more distantly related from the other members of this superfamily.

Based upon the alignment and the motifs derived from MEME algorithm, we created a PROSITE regular expression from a conserved region and used it to search the protein database as a pattern seed by PHI-BLAST program (Zhang et al. (1998) Nucleic Acids Res. 26:3986-3990). This algorithm has been reported to be very useful for identify genes that share a pattern that is indicative of functional relationship. By this approach we retrieved 98 sequences that contained a common pattern—[ILM]-[RK]-X(2)-[VLI]-[PGA]-X(10,11)-[RX]-X(2)-[IVLI]-X(7)-[IVLIM]-X(6)-[WFY]- which were above the threshold of 0.001 and displayed very significant E-values. Of these the HIR proteins, stomatins and other membrane-associated proteins had E values <10⁻⁴, and prohibitins had relatively higher E values (E=0.003-10.0). Considering the size of the database searched and the functionally divergent sequences in this superfamily, the seed pattern used has been shown by this search method to be highly effective at successfully retrieving members for each of the three families of this superfamily. Consequently, this pattern possibly represents a signature for this newly identified PID superfamily. This signature pattern is found in stomatin amino acid sequence of SEQ ID NO:2 from amino acid residues 154 to 188.

In the PROSITE dictionary, stomatin (Band 7) signature is in part present in HIR proteins, having diverged by about 40% (at seven out of sixteen positions in the stomatin signature, when conservative substitutions are considered. The PID superfamily signature we identified partially overlaps the PROSITE Band 7 signature, however the PID signature predicts all superfamily members and not just the stomatins. The PID signature actually overlaps with Motif 1 region as discovered by MEME algorithm. Although Motif 1 is not well conserved in HFLK/HFLC proteins, the PID signature is conserved in HFLK/HFLC, indicating that these proteins are distant members of this superfamily.

The sequence and structural similarities of plant HIR proteins with prohibitins, stomatins and other integral membrane proteins, some of which, in particular stomatins, regulate ion channel function, suggests that the HIR proteins are involved in hypersensitive reaction and cell death through the regulation of ion channel activity. The C-terminal region of stomatin is very rich in alpha-helical content and has been postulated to act as a plug to regulate potassium ion channels (Stewart et al. (1993) Biochimica et Biophysica Acta. 1225:15-25; Stewart (1997) Int. J. Biochem. Cell Biol. 29:271-274). The HIR proteins also have C-termini with very high in helical content, suggesting a similar structure and function of this region to that of the stomatin C-termini.

In conclusion, a supergene family named PID (Proliferation, Ion, and Death) has been identified. Members of the family include prohibitins and stomatins and the HIR genes. Proteins of this superfamily are involved in diverse functions, but their structural similarity suggests a conserved molecular mechanism.

272 V

| Ingredient | Amount | Unit |
|---|---|---|
| D-I H₂O | 950.000 | Ml |
| MS Salts (GIBCO 11117-074) | 4.300 | G |
| Myo-Inositol | 0.100 | G |
| MS Vitamins Stock Solution ## | 5.000 | Ml |
| Sucrose | 40.000 | G |
| Bacto-Agar @ | 6.000 | G |

Directions:
@ = Add after bringing up to volume
Dissolve ingredients in polished D-I H₂O in sequence
Adjust to pH 5.6
Bring up to volume with polished D-I H₂O after adjusting pH
Sterilize and cool to 60° C.
## = Dissolve 0.100 g of Nicotinic Acid; 0.020 g of Thiamine.HCL;
0.100 g of Pyridoxine.HCL; and 0.400 g of Glycine in 875.00 ml of
polished D-I H₂O in sequence.
Bring up to volume with polished D-I H₂O. Make in 400 ml portions.
Thiamine.HCL & Pyridoxine.HCL are in Dark Desiccator. Store for one
month, unless contamination or precipitation occurs, then make fresh
stock.
Total Volume (L) = 1.00

288 J

| Ingredient | Amount | Unit |
|---|---|---|
| D-I H₂O | 950.000 | Ml |
| MS Salts | 4.300 | G |
| Myo-Inositol | 0.100 | G |
| MS Vitamins Stock Solution ## | 5.000 | Ml |
| Zeatin .5 mg/ml | 1.000 | Ml |
| Sucrose | 60.000 | G |
| Geirite @ | 3.000 | G |
| Indoleacetic Acid 0.5 mg/ml # | 2.000 | Ml |
| 0.1 mM Abscisic Acid | 1.000 | Ml |
| Bialaphos 1 mg/ml ft | 3.000 | Ml |

Directions:
@ = Add after bringing up to volume
Dissolve ingredients in polished D-I H₂O in sequence
Adjust to pH 5.6
Bring up to volume with polished D-I H₂O after adjusting pH
Sterilize and cool to 60° C.
Add 3.5 g/L of Gelrite for cell biology.
## = Dissolve 0.100 g of Nicotinic Acid; 0.020 g of Thiamine.HCL;
0.100 g of Pyridoxine.HCL; and 0.400 g of Glycine in 875.00 ml of
polished D-I H₂O in sequence.
Bring up to volume with polished D-I H₂O. Make in 400 ml portions.
Thiamine.HCL & Pyridoxine.HCL are in Dark Desiccator. Store for one
month, unless contamination or precipitation occurs, then make fresh
stock.
Total Volume (L) = 1.00

560 R

| Ingredient | Amount | Unit |
|---|---|---|
| D-I Water, Filtered | 950.000 | Ml |
| CHU (N6) Basal Salts (SIGMA C-1416) | 4.000 | G |
| Eriksson's Vitamin Mix (1000X SIGMA-1511 | 1.000 | Ml |
| Thiamine.HCL 0.4 mg/ml | 1.250 | Ml |
| Sucrose | 30.000 | G |
| 2,4-D 0.5 mg/ml | 4.000 | Ml |
| Gelrite @ | 3.000 | G |
| Silver Nitrate 2 mg/ml # | 0.425 | Ml |
| Bialaphos 1 mg/ml # | 3.000 | Ml |
| Directions: | | |
| @ = Add after bringing up to volume | | |
| # = Add after sterilizing and cooling to temp. | | |
| Dissolve ingredients in D-I H₂O in sequence | | |
| Adjust to pH 5.8 with KOH | | |
| Bring up to volume with D-I H₂O | | |
| Sterilize and cool to room temp. | | |
| Total Volume (L) = 1.00 | | |

560 Y

| Ingredient | Amount | Unit |
|---|---|---|
| D-I Water, Filtered | 950.000 | Ml |
| CHU (N6) Basal Salts (SIGMA C-1416) | 4.000 | G |
| Eriksson's Vitamin Mix (1000X SIGMA-1511 | 1.000 | Ml |
| Thiamine.HCL 0.4 mg/ml | 1.250 | Ml |
| Sucrose | 120.000 | G |
| 2,4-D 0.5 mg/ml | 2.000 | Ml |
| L-Proline | 2.880 | G |
| Geirite @ | 2.000 | G |
| Silver Nitrate 2 mg/ml # | 4.250 | Ml |
| Directions: | | |
| @ = Add after bringing up to volume | | |
| # = Add after sterilizing and cooling to temp. | | |
| Dissolve ingredients in D-I H₂O in sequence | | |
| Adjust to pH 5.8 with KOH | | |
| Bring up to volume with D-I H₂O | | |
| Sterilize and cool to room temp. | | |
| **Autoclave less time because of increased sucrose** | | |
| Total Volume (L) = 1.00 | | |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated polypeptide selected from the group consisting of:
a) a polpypeptide comprising an amino acid sequence set forth in SEQ ID NO:2;
b) a polypeptide encoded by a nucleotide sequence comprising the sequence set forth in SEQ ID NO:1;
c) a polypeptide encoded by a nucleotide sequence that hybridizes under stringent conditions to a nucleotide sequence comprising the sequence set forth in SEQ ID NO:1, wherein said polypeptide retains stomatin-like activity;
d) a polypeptide having at least 70% sequence identity to the amino acid sequence of SEQ ID NO:2, wherein said sequence retains stomatin-like activity; and,
e) a polypeptide having at least 25 contiguous amino acids of SEQ ID NO:2, wherein said sequence retains stomatin-like activity.
